(19) United States
(12) Patent Application Publication
(10) Pub. No.: US 2025/0003856 A1
(43) Pub. Date: Jan. 2, 2025

(54) SYSTEMS AND METHODS TO DETERMINE BIOT COEFFICIENT AND EFFECTIVE STRESS DEPENDENCE COEFFICIENT IN ROCK

(71) Applicant: ARAMCO SERVICES COMPANY, Houston, TX (US)

(72) Inventors: Jilin Jay Zhang, Houston, TX (US); Hui-Hai Liu, Katy, TX (US); Jewel Duncan, Houston, TX (US)

(73) Assignee: ARAMCO SERVICES COMPANY, Houston, TX (US)

(21) Appl. No.: 18/343,684

(22) Filed: Jun. 28, 2023

(51) Int. Cl. G01N 15/08; G01N 33/24

(52) U.S. Cl. CPC G01N 15/082; G01N 33/241

(57) ABSTRACT

Methods and systems are disclosed. The methods may include obtaining, from a subterranean region of interest, a rock sample having a rock type and defining a sequence of pore pressure, confining stress (PPCS) pairs such that a sequence of effective stresses monotonically changes. The methods may further include determining a sequence of permeabilities by subjecting the rock sample to the sequence of PPCS pairs and determining a relationship between the sequence of PPCS pairs and the sequence of permeabilities. The methods may further still include determining a parameter using the relationship and a permeability model, where the permeability model includes the parameter and determining an in situ permeability for an in situ rock in the subterranean region of interest using, at least in part, the parameter and the permeability model, where the in situ rock is of the rock type.

### BACKGROUND

Rock within a subterranean region of interest may be composed of grains and pores. Fluids, such as carbon dioxide, water, brine, and hydrocarbons, may permeate those pores. Further, the fluids may flow through the pores of the rock. A measure of how easily the fluids flow is known as permeability. As geological conditions, such as the deposition of overburden rock, occur within the subterranean region of interest, increased effective stress applied to the rock may reduce permeability. Reduced permeability may limit fluid flow to make fluid extraction more difficult. As such, quantifying the permeability of rock under various stress conditions may be useful in predicting fluid production rates, specifically hydrocarbon production rates.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method. The method includes obtaining, from a subterranean region of interest, a rock sample having a rock type and defining a sequence of pore pressure, confining stress (PPCS) pairs such that a sequence of effective stresses monotonically changes. The method further includes determining a sequence of permeabilities by subjecting the rock sample to the sequence of PPCS pairs and determining a relationship between the sequence of PPCS pairs and the sequence of permeabilities. The method further still includes determining a parameter using the relationship and a permeability model, where the permeability model includes the parameter, and determining an in situ permeability for an in situ rock in the subterranean region of interest using, at least in part, the parameter and the permeability model, where the in situ rock is of the rock type.

In general, in one aspect, embodiments relate to a system. The system includes a hydrostatic permeability system configured to subject a rock sample to a sequence of PPCS pairs. The system further includes a computer system configured to receive the sequence of PPCS pairs such that a sequence of effective stresses monotonically changes and determine a sequence of permeabilities following the rock sample being subjected to the sequence of PPCS pairs using the hydrostatic permeability system. The computer system is further configured to determine a relationship between the sequence of PPCS pairs and the sequence of permeabilities and determine a parameter using the relationship and a permeability model, where the permeability model includes the parameter. The computer system is further still configured to determine an in situ permeability for an in situ rock in a subterranean region of interest using, at least in part, the parameter and the permeability model, where the in situ rock is of a rock type.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 illustrates a subterranean region of interest in accordance with one or more embodiments.

FIGS. 2A and 2B illustrate rock in accordance with one or more embodiments.

FIG. 3 illustrates permeability hysteresis in accordance with one or more embodiments.

FIG. 4 shows a sequence of pore pressure, confining stress pairs in accordance with one or more embodiments.

FIG. 5 illustrates a coring system in accordance with one or more embodiments.

FIG. 6 illustrates a hydrostatic permeability system in accordance with one or more embodiments.

FIGS. 7A and 7B show relationships in accordance with one or more embodiments.

FIG. 8 shows a flowchart in accordance with one or more embodiments.

FIG. 9 illustrates a computer in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a pore pressure” includes reference to one or more of such pressures.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

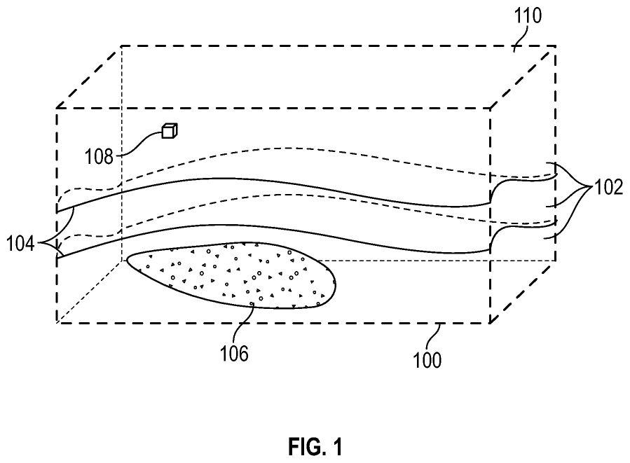

In the following description of FIGS. 1-9, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

Methods and systems are disclosed to determine parameters, such as a Biot coefficient and an effective stress dependence coefficient, of a rock. A rock sample may be extracted from a subterranean region of interest. The rock sample may be subjected to a sequence of pore pressure, confining stress (PPCS) pairs using a hydrostatic permeability system to determine a sequence of permeabilities. The sequence of PPCS pairs and the sequence of permeabilities may be used to determine the Biot coefficient and/or the effective stress dependence coefficient assuming a permeability model. The parameters may then be used to determine the in situ permeability of in situ rock within the subterranean region of interest. The permeability of the in situ rock may be used to aid in determining hydrocarbon production rates.

Turning to FIG. 1, FIG. 1 illustrates a subterranean region of interest (100) in accordance with one or more embodiments. The subterranean region of interest (100) may consist of layers of rock (102) separated by geological boundaries (104). Some layers of rock (102) may reside above a hydrocarbon reservoir (106). Any layer of rock (102) that resides above the hydrocarbon reservoir (106), whether the layer of rock (102) is directly above the hydrocarbon reservoir (106) or just below the surface (110), may be referred to as “overburden rock.” The hydrocarbon reservoir (106) may be rock from which hydrocarbons are generated (hereinafter “source rock”) or may be adjacent to source rock (102). For example, an unconventional hydrocarbon reservoir (106) may be shale rock. Hereinafter, any rock (102) within a subterranean region of interest (100) may be denoted “in situ rock” or simply “rock.”

In situ rock (102) at different positions (108) within the subterranean region of interest (100) may be in different stress states. Note that stress is a normalized measure of force, in particular a force per cross-sectional area. The stress state of in situ rock (102) at each position (108) may be caused, in part, by the types and degree of geological conditions and/or manmade activities affecting the in situ rock (102). Geological conditions may include, but are not limited to, the weight of overburden rock, tectonics, thermal processes, and glacial rebound. Manmade activities may include, but are not limited to, drilling a well, well completion strategies, hydrocarbon or other fluid recovery, and mining tunnels. For example, in situ rock (102) deep within the subterranean region of interest (100) may present a higher stress state relative to in situ rock (102) from similar rocks of the same rock type near the surface (110) due to the weight of overburden rock. In another example, drilling a well within the subterranean region of interest (100) may result in cracked or weakened in situ rock (102) that presents a lower stress state relative to the stress state of the in situ rock (102) at that position (108) prior to drilling the well.

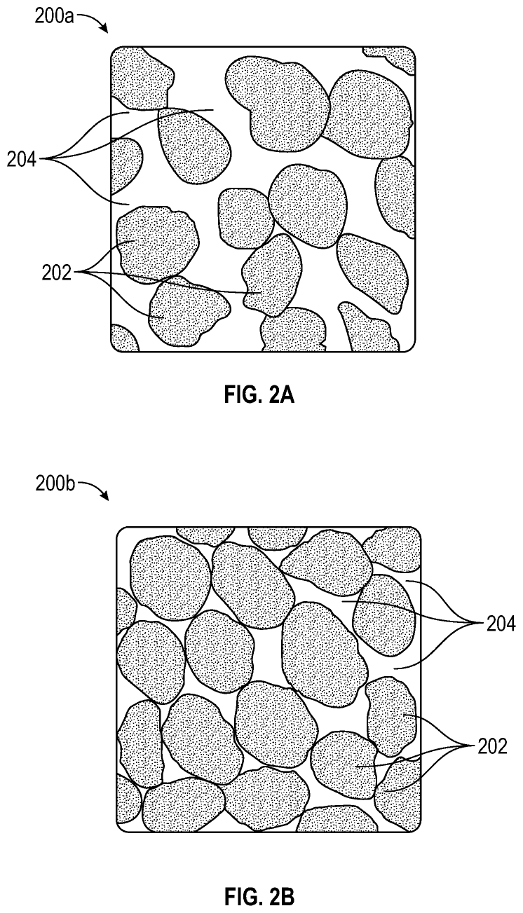

The stress state of in situ rock (102) may affect each constituent of in situ rock (102). Turning to FIGS. 2A and 2B, the constituents of in situ rock (200a, b) may include grains (202) and pores (204). The grains (202) may be a material made up of, but not limited to, sandstone, carbonate, or shale. The material of the grains (202) may be a key consideration in determining which “rock type” the in situ rock (200a, b) is. The pores (204) are voids between the grains (202). The pores (204) may be saturated with fluid. The fluid may be, but is not limited to, air, natural gas, water, brine, other hydrocarbons, or any mixture thereof. For example, the rock (200a, b) of an unconventional hydrocarbon reservoir (106) may consist of shale grains (202) with hydrocarbon-saturated pores (204). Further, rock (200a, b) with hydrocarbon-saturated pores (204) may be considered source rock.

The material of the grains (202) and the fluid saturating the pores (204) of the rock (200a, b) may dictate the physical properties, mechanical behavior, and parameters of the rock (200a, b). Physical properties include, but are not limited to, porosity, permeability, pore pressure, confining stress, and effective stress. Porosity is defined as the fraction of the volume of the rock (200a, b) that is occupied by the pores (204). For example, unconventional hydrocarbon reservoirs (106) may have a low porosity under 5%. Permeability may be closely related to porosity. Permeability is a measure of how easily fluid flows through the rock (200a, b). The degree of connection between the pores (204) and the viscosity of the fluid saturating the pores (204) may constrain permeability. Thus, FIG. 2A may depict rock (200a) with high porosity and high permeability, while FIG. 2B may depict rock (200b) with low porosity and low permeability. Note that high porosity, low permeability rock and low porosity, high permeability rock can also exist.

Pore pressure, another physical property of rock (200a, b), may be the pressure associated with the fluids saturating the pores (204) of the rock (200a, b). In some embodiments, the pore pressure may be considered hydrostatic or at equilibrium. Confining stress may be the stress caused by overburden rock (200a, b). As such, confining stress may be alternatively referred to as “overburden stress.” Effective stress may be a function of the pore pressure and confining stress acting on the rock (200a, b). In some embodiments, the effective stress σₑ may be modeled as:

$${σ}_{e} = {σ}_{c} - β {p}_{p} , \qquad Equation ( 1 )$$

where σ_{c} is confining stress, pₚ is pore pressure, and β is a Biot coefficient. The effective stress σₑ may be thought of as the stress that controls the mechanical behavior of the rock (200a, b) where the confining stress cc is supported by the grains (202) and a portion of the pore pressure pₚ supports the grains (202). However, a person of ordinary skill in the art will appreciate that effective stress σₑ may be modeled different than Equation (1). The effective stress σₑ may quantify the stress state of the rock (200a, b).

The Biot coefficient β as well as an effective stress dependence coefficient α may be considered members of the group of parameters characterizing the rock (200a, b). The Biot coefficient β may also be obtained from a measure of the bulk modulus of the rock (200a, b) void of fluid relative to the bulk modulus of only the grains (202) of the rock (200a, b). Further, the Biot coefficient β may range between zero and one. The effective stress dependence coefficient α may be a constant that, in part, relates effective stress σₑ to permeability k. For example, permeability k may be modeled as:

$$k = {k}_{0} exp ( - α {σ}_{e} ) = {k}_{0} exp ( - α ( {p}_{c} - β {p}_{p} ) ) , \qquad Equation ( 2 )$$

where k₀ is the permeability when the effective stress σₑ is zero. Based on Equation (2), permeability k may exponentially decrease as effective stress σₑ increases. As such, the stress state of in situ rock (200a, b) may affect the in situ permeability k of the rock (200a, b).

Turning to the mechanical behavior of rock (200a, b), the material of the grains (202) may present elastic behavior or plastic behavior depending on the size of the applied force. The grains (202) may present elastic behavior when small forces are applied. In the elastic range, the grains (202) will return to their undeformed shape following the removal of a small force. The grains (202) may present plastic behavior when large forces are applied. In the plastic range, the grains (202) will maintain a deformed shape following the removal of a large force. However, in either the elastic or plastic range, the framework made up of the grains (202) may also consolidate due to an applied force.

The fluid-saturated pores (204) of rock (200a, b) may present viscoelastic behavior in that the fluid presents both viscous behavior and elastic behavior. Viscous behavior may be quantified by the viscosity of the fluid saturating the pores (204). Viscosity is a measure of the rate of resistance of the fluid to deform when a force is applied or removed.

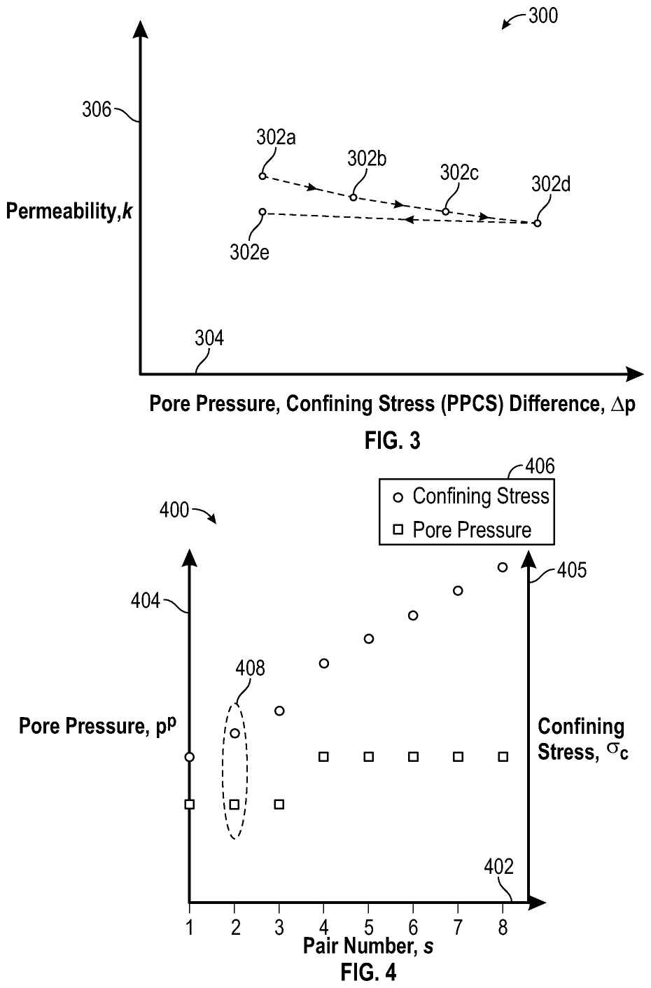

Rock (200a, b) may present a phenomenon known as hysteresis. FIG. 3 illustrates permeability hysteresis (300) in accordance with one or more embodiments. The abscissa (304) presents PPCS difference Δp (hereinafter also “difference”). The ordinate (306) presents permeability k. The permeability, difference pairs (302a-e) may be determined for a rock sample in a laboratory setting. For example, a test, such as a static mechanical test, may be applied to the rock sample in a laboratory setting to determine permeability, difference pairs (302a-e) such as those shown in FIG. 3.

Permeability hysteresis (300) describes a phenomenon where the determined values of permeability k depend on the history of stresses experienced by the sample, not just the current stress state. The application of stress may be referred to as “loading.” The removal of stress may be referred to as “unloading.” Referring to FIG. 3, stress is described in terms of the PPCS difference Δp. The first permeability, difference pair (302a) presents an initial permeability k at an initial PPCS difference Δp. As larger PPCS differences Δp are applied to the rock sample (i.e., the rock sample is loaded), permeability k decreases. However, upon unloading to return to the initial PPCS difference Δp, the permeability k has now shifted lower than the initial permeability k as shown by the fifth permeability, difference pair (302e).

Permeability hysteresis (300) may be avoided during a test if a sequence of PPCS pairs applied to a rock sample during the test is defined such that only loading or only unloading of a rock sample occurs. For example, if only loading of a rock sample occurs, only the first permeability, difference pair (302a) through the fourth permeability, difference pair (302d) in FIG. 3 may occur during the testing of the rock sample. FIG. 4 illustrates a sequence of PPCS pairs (400) where only loading occurs in accordance with one or more embodiments. The abscissa (402) presents the ordering of the sequence of PPCS pairs (400) designated by pair number s one through eight. The left ordinate (404) presents the value of each pore pressure pₚ and the right ordinate (405) presents the value of each confining stress σ_{c}. Note that while pressure and stress are different, the units of pressure and stress may be the same.

As shown by the key (406), each square denotes a value of a pore pressure pₚ and each circle denotes a value of a confining stress σ_{c} among the sequence of PPCS pairs (400). The rock sample may be subjected to each PPCS pair (408) sequentially in order as designated by the pair number s along the abscissa (402).

The sequence of PPCS pairs is defined such that only loading or only unloading of the rock sample occurs. Only loading occurs when the effective stress for each PPCS pair (408), where effective stress σₑ may be defined by Equation (1), increases relative to the effective stress σₑ for the previous PPCS pair (408) within the sequence of PPCS pairs (400). As such, the sequence of effective stresses monotonically increases. Only unloading occurs when the effective stress for each PPCS pair (408) decreases relative to the effective stress σₑ for the previous PPCS pair (408) within the sequence of PPCS pairs (400). As such, the sequence of effective stresses monotonically decreases. In the context of this disclosure, “monotonically changes” is used to describe a sequence of effective stresses that either monotonically decreases or monotonically increases. A sequence of effective stresses that monotonically changes, thus, avoids permeability hysteresis (300) as only loading or unloading of the rock sample occurs. A person of ordinary skill in the art will appreciate that the sequence of PPCS pairs (400) may be any sequence such that the sequence of effective stresses monotonically changes assuming an equation other than Equation (1) that relates at least pore pressure pₚ, confining stress σ_{c}, and effective stress σₑ.

In the world outside the laboratory, monotonically increasing loading of rock may occur during hydrocarbon production where pore pressure pₚ decreases while confining stress σ_{c} remains constant. In contrast, monotonically decreasing rock loading (or “unloading”) may occur during carbon dioxide sequestration where carbon dioxide is pumped into one or more layers of rock (102) causing pore pressure pₚ to increase while confining stress σ_{c} remains constant.

Returning to FIG. 4, while FIG. 4 shows eight ordered PPCS pairs (408) within the sequence of PPCS pairs (400), any number of PPCS pairs (408) may make up the sequence of PPCS pairs (400). Further, any values of pore pressures pₚ and confining stresses Qc may be defined for each of the PPCS pairs (408) as long as the resulting sequence of effective stresses monotonically changes. However, it may be beneficial to maintain values of pore pressures pₚ above 10 megapascals (MPa) (or approximately 1500 pounds per square inch (psi)) to minimize Knudsen diffusion when determining permeability k.

Table 1 below is another sequence of PPCS pairs (400) where the sequence of effective stresses monotonically increases.

TABLE 1

| | | |
|---|---|---|
| Pair Number, s | pₚ MPa (psi) | σ_{c} MPa (psi) |

| | | | |
|---|---|---|---|
| 1 | 13.8 | (2000) | 20.7 (3000) |
| 2 | 13.8 | (2000) | 24.1 (3500) |
| 3 | 13.8 | (2000) | 27.6 (4000) |
| 4 | 20.7 | (3000) | 34.5 (5000) |
| 5 | 24.1 | (3500) | 37.9 (5500) |
| 6 | 27.6 | (4000) | 41.4 (6000) |
| 7 | 31 | (4500) | 44.8 (6500) |
| 8 | 34.5 | (5000) | 48.3 (7000) |

To apply the sequence of PPCS pairs (400) to a rock sample in a laboratory setting, a rock sample must first be obtained from a subterranean region of interest (100). A rock sample may be obtained from a subterranean region of interest (100) using a rock sample extraction tool. In some embodiments, the rock sample extraction tool may be a coring system that is used to drill a well and retrieve a rock core in parallel.

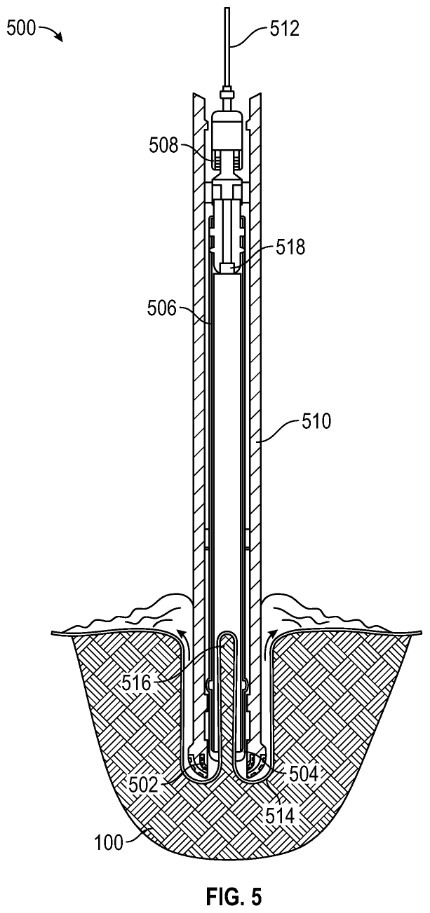

FIG. 5 illustrates a coring system (500) in accordance with one or more embodiments. The coring system (500) may consist of a core bit (502), core catcher (504), inner barrel (506), swivel (508), and outer barrel (510) among other components. In some embodiments, the core catcher (504) and inner barrel (506) may be suspended by the swivel (508) while the outer barrel (510) may mate with the core bit (502) and a drillstring (512). The coring system (500) may be deployed within a subterranean region of interest (100) using the drillstring (512). As the core bit (502) rotates to drill a well (514) within the subterranean region of interest (100), the core bit (502) simultaneously retrieves a rock core (516). The rock core (516) may be held in place by the core catcher (504) and stored in the inner barrel (506). Once the rock core (516) reaches the end (518) of the inner barrel (506), an overshot (not shown) may be deployed downhole to retrieve the inner barrel (506) that contains the rock core (516). Once the inner barrel (506) is on the surface (110), the rock core (516) is removed from the inner barrel (506).

The extracted rock core (516) may be up to 15 centimeters in diameter and approximately ten meters long. To prepare the rock core (516) for testing in a laboratory setting, the rock core (516) may be cut and ground into core plugs. A core plug may be a few centimeters in diameter and approximately five centimeters long, though other shapes and dimensions may be used. Further, a core plug may be cut and ground along a particular axis, such as parallel or perpendicular to an axis of the well (514) being drilled. Hereinafter, a core plug will be referred to as simply a “rock sample.”

The rock sample may be dried to remove fluids, such as water and hydrocarbons. In some embodiments, the rock sample may be placed in a vacuum oven to remove the fluids. The rock sample may also be cyclically pre-stressed (i.e., loading and unloaded repeatedly) to remove inelastic deformation. In some embodiments, cyclical pre-stressing may occur over a period of days.

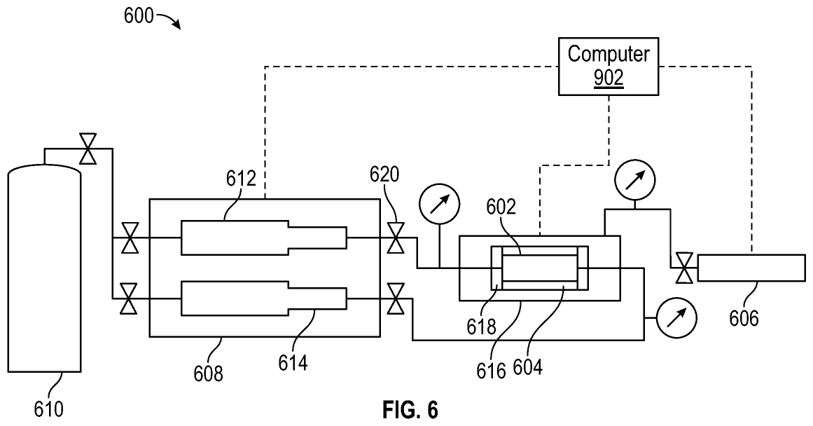

The rock sample may be cyclically pre-stressed as well as subjected to the previously defined sequence of PPCS pairs (400) using a hydrostatic permeability system. FIG. 6 illustrates a hydrostatic permeability system (600) in accordance with one or more embodiments. Hereinafter, the application of the sequence of PPCS pairs (400) to the rock sample (602) may be simply referred to as a “test” or “testing.” Prior to cyclical pre-stressing or testing, the rock sample (602) is wrapped in a jacket (604), placed between two endcaps (618), and housed within a confining cell (616). In some embodiments, the jacket (604) may be a hollow Viton sleeve. A pressure generator (606) may be connected to the confining cell (616) and configured to provide a fluid to the confining cell (616) to control confining stress σ_{c}. A gas pump system (608) may be connected to each end of the rock sample (602) via the upstream reservoir 612 and the downstream reservoir 614 and configured to uniquely control pore pressure pₚ at either end of the rock sample (602) when valves (620) are closed. To control pore pressure pₚ, a gas tank (610) may supply helium, nitrogen, or other gas into the pores (204) of the rock sample (602) via an upstream reservoir (612) and/or a downstream reservoir (614) within the gas pump system (608). As such, wrapping the rock sample (602) in a jacket (604) between two endcaps (618) housed in a confining cell (616) may ensure the helium, nitrogen, or other gas supplied by the gas tank (610) does not communicate with the fluid that controls confining stress σ_{c}.

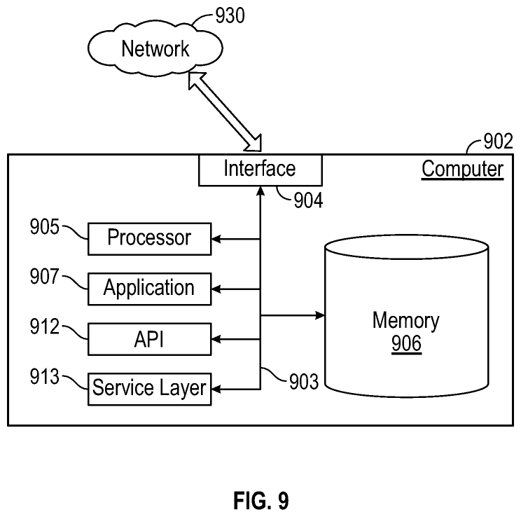

The upstream reservoir (612) and downstream reservoir (614) may also be used to recover gas from the rock sample (602) depending on the mode of operation of the gas pump system (608). For example, if a constant pore pressure pₚ is applied to the rock sample (602), both the upstream reservoir (612) and downstream reservoir (614) may supply gas to the rock sample (602). However, if a pressure pulse is applied to the rock sample (602), the upstream reservoir (612) at a high pressure may supply gas while the downstream reservoir (614) at a low pressure recovers the gas that passed though the rock sample (602). In some embodiments, various parts of the hydrostatic permeability system (600) may be communicably coupled to a computer (902) as will be described in reference to FIG. 9. The computer (902) may be configured to control and/or collect data from the pressure generator (606), the gas tank (610), and/or the gas pump system (608).

During testing, each PPCS pair (408) among the sequence of PPCS pairs (400) is applied to the rock sample (602) statically in series. While the rock sample (602) is subjected to each PPCS pair (408), permeability k is determined. Following testing, a sequence of permeabilities is determined.

Each permeability k may be determined using indirect methods, such as a pressure pulse decay method or steady-state Darcy flow method. The pressure pulse decay method may take on the order of hours. The steady-state Darcy flow method may take on the order of hours or even days. In brief, the pressure pulse decay method may rely on the gas pump system (608) to generate small pressure pulses at the upstream reservoir (612) that travel through a test sample to the downstream reservoir (614). In some embodiments, each pressure pulse may be small to minimize changes in pore pressure pₚ. For each pressure pulse, the first transient pressure at the upstream reservoir (612) and the second transient pressure at the downstream reservoir (614) for each PPCS pair (408) may be fit to a permeability-pressure model to determine permeability k.

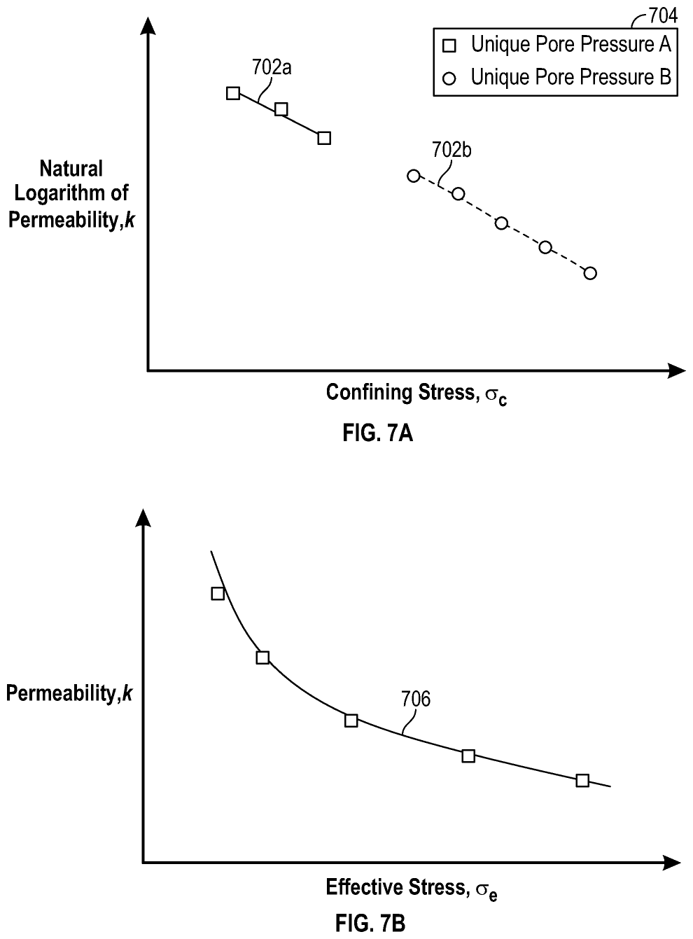

The sequence of PPCS pairs (400) and the sequence of permeabilities may be used to determine a relationship. In some embodiments, the relationship may be one or more linear lines (702a, b) fit to a natural logarithm of the sequence of permeabilities versus a sequence of confining stresses for each unique pore pressure pₚ among the sequence of PPCS pairs (400) as illustrated in FIG. 7A. FIG. 7A presents two linear lines (702a, b) as there are two unique pore pressures pₚ generically denoted A and B as shown by the key (704). Each linear line (702a, b) may take the form:

$$y = a x + b , \qquad Equation ( 3 )$$

where y is the natural logarithm of the sequence of permeabilities, x is the sequence of confining stresses, and a and b are estimated constants.

In other embodiments, the relationship may be an exponential line (706) fit to the sequence of permeabilities versus the sequence of effective stresses as illustrated in FIG. 7B. In some embodiments, the Biot coefficient β may be previously known such that the sequence of effective stresses may be determined from the sequence of PPCS pairs (400) using Equation (1). In some embodiments, there may be only one unique pore pressure pₚ. The exponential line (706) may take the form:

$$y = c {e}^{- dx} , \qquad Equation ( 4 )$$

where y is the sequence of permeabilities, x is the sequence of effective stresses, and c and d are estimated constants.

In still other embodiments, a curve may be fit to the sequence of permeabilities versus a sequence of pore pressures among the sequence of PPCS pairs (400) for each PPCS difference Δp. Any relationship, such as a linear or exponential relationship, may be fit to any manipulation of the sequence of PPCS pairs (400) and the sequence of permeabilities without departing from the scope of the disclosure.

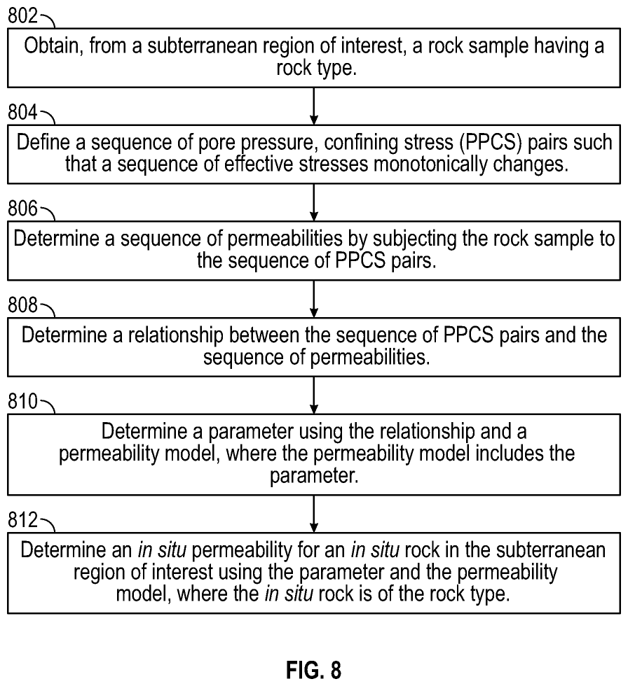

FIG. 8 describes a method in accordance with one or more embodiments. In step 802, a rock sample (602) is obtained from a subterranean region of interest (100). The rock sample (602) is obtained from the subterranean region of interest (100) using a rock sample extraction tool. In some embodiments, the rock sample extraction tool may be a coring system (500) as described in FIG. 5. The rock sample (602) is of a rock type, such as sandstone, carbonate, or shale, among others. The rock sample (602) may also be source rock where the pores (204) are saturated with natural gas or other hydrocarbons.

In step 804, a sequence of PPCS pairs (400) is defined. The order of the sequence of PPCS pairs (400) is defined such that a sequence of effective stresses monotonically changes. For example, assume Equation (1) defines the relationship between effective stress σₑ, pore pressure pₚ, and confining stress σ_{c}. In these embodiments, the sequence of PPCS pairs (400) may be defined such that for each PPCS pair (408), the effective stress determined using Equation (1) increases relative to the effective stress determined using the previous PPCS pair (408). In these embodiments, the sequence of effective stresses monotonically increases. Because, in some embodiments, the Biot coefficient β may be unknown when the sequence of PPCS pairs (400) is defined, the following relations may be used to ensure the sequence of effective stresses monotonically increases for the sequence of PPCS pairs (400):

$$Δ {p}^{[ s + 1 ]} - Δ {p}^{[ s ]} \geq 0 , and \qquad Equation ( 5 )$$

$${p}_{p}^{[ s + 1 ]} - {p}_{p}^{[ s ]} \geq 0. \qquad Equation ( 6 )$$

In Equations (5) and (6), Δₚ=σ_{c}−pₚ. As previously described relative to FIG. 4, s denotes the pair number in the sequence of PPCS pairs (400) where s=1, 2, . . . , n and n is the total number of PPCS pairs (408) in the sequence of PPCS pairs (400).

In step 806, a sequence of permeabilities is determined for the rock sample (602). A permeability k is determined for each PPCS pair (408) in the sequence of PPCS pairs (400) in series. The rock sample (602) may be placed in a hydrostatic permeability system (600) as previously described in FIG. 6. With the rock sample (602) in the hydrostatic permeability system (600), the first PPCS pair (408) in the sequence of PPCS pairs (400) is applied to the rock sample (602). In some embodiments, a method to determine permeability k is concurrently applied to the rock sample (602). The method to determine permeability k may be, but is not limited to, a pressure pulse decay method or a steady-state Darcy flow method. Once the method to determine the permeability k is complete, the second PPCS pair (408) in the sequence of PPCS pairs (400) is applied to the rock sample (602) and a method to determine permeability k applied to the rock sample (602). This process is repeated until a permeability k has been determined for each PPCS pair (408) within the sequence of PPCS pairs (400). In some embodiments, the same method to determine permeability k for each PPCS pair (408) is used. In other embodiments, different methods may be used to determine permeability k within the sequence of permeabilities.

In step 808, a relationship is determined between the sequence of PPCS pairs (400) defined in step 804 and the sequence of permeabilities determined in step 806. In some embodiments, the actual sequence of PPCS pairs (400) applied to the rock sample (602) using the hydrostatic permeability system (600) may be used in place of the sequence of PPCS pairs (400) defined in step 804 as the two sequences may be slightly different. In some embodiments, the relationship may be determined as described in FIGS. 7A and 7B.

In step 810, a parameter is determined using the relationship from step 808 and a permeability model. The parameter may be extracted from the relationship assuming the permeability model. For example, assume the permeability model is presented in Equation (2). Assume the relationship is in the form presented in Equation (3) and shown in FIG. 7A. In these embodiments, the effective stress dependence coefficient α is a parameter. In some embodiments, the effective stress σₑ within Equation (2) may be further defined by Equation (1). In these embodiments, the Biot coefficient β is another parameter. Then, the effective stress dependence coefficient α may be determined from M linear lines (702a, b) by:

$$α = - \frac{{\sum }_{m = 1}^{M} {a}_{m}}{M} , \qquad Equation ( 7 )$$

where the slope of the first linear line (702a) is a₁, the slope of the second linear line (702b) is a₂, etc. Following determination of the effective stress dependence coefficient α using Equation (7), the Biot coefficient β may be determined from pairs of the M linear lines (702a, b) where the Biot coefficient β for each ij pair may be:

$${β}_{ij} = \frac{{b}_{j} - {b}_{i}}{{α}_{j} {P}_{p , j} - {α}_{i} {P}_{p , i}} , \qquad Equation ( 8 )$$

where the y-intercept of the first linear line (702a) is bᵢ and the y-intercept of the second linear line (702b) is bⱼ. The term βᵢⱼ for all pairs Q may then be averaged to determine β where:

$$β = - \frac{{\sum }_{q = 1}^{Q} {β}_{ij , Q}}{Q} . \qquad Equation ( 9 )$$

In another example, the Biot coefficient β may be assumed. The effective stress σₑ as given in Equation (1) may then be determined for each PPCS pair (408) using the assumed Biot coefficient β to determine a sequence of effective stresses. An exponential relationship in the form of Equation (4) may then be determined for the sequence of permeabilities and the sequence of effective stresses, as presented in FIG. 7B, to estimate the coefficients c and d. The initial permeability k₀ and the effective stress dependence coefficient α from Equation (2) may then be approximated by c and d, respectively.

In yet another example, a linear relationship may be determined for the natural logarithm of the sequence of permeabilities versus the pore pressures pₚ among the sequence of PPCS pairs (400) for each unique PPCS difference Δp among the sequence of PPCS pairs (400). A new linear relationship may then be determined from the y-intercepts bₘ of the linear relationships versus the PPCS differences Δp. The effective stress dependence coefficient α from Equation (2) may then be approximated by the slope a′ of the new linear relationship where:

$$α = - {a}^{′} . \qquad Equation ( 10 )$$

The Biot coefficient β may then be determined using the effective stress dependence coefficient α and the slopes am of the linear relationships where:

$$\frac{{\sum }_{m = 1}^{M} {a}_{m}}{M} = - α ( 1 - β ) . \qquad Equation ( 11 )$$

A person of ordinary skill in the art will appreciate that effective stress models other than Equation (1) may be used and permeability models other than Equation (2) may be used. Further, a person of ordinary skill in the art will appreciate that relationships other than linear and exponential relationships, as presented in Equations (3) and (4), may be determined from the sequence of permeabilities and the sequence of PPCS pairs (400) to determine a parameter.

In step 812, the parameter from step 810 may be used to determine in situ permeability k for in situ rock (200a, b) in the subterranean region of interest (100). In some embodiments, the effective stress model presented in Equation (1) and the permeability model presented in Equation (2) may be used to determine in situ permeability k of in situ rock (200a, b). In situ pore pressure pₚ wireline formation tests, pressure transient well tests, and in situ confining stress ac may be determined from well logs, such as density logs. The Biot coefficient β and/or the effective stress dependence coefficient α may be determined using steps 802 through 810. In some embodiments, the initial permeability k₀ may also be determined following steps 802 through 810. In other embodiments, the initial permeability k₀ may be determined using another test in a laboratory setting. The values of in situ pore pressure pₚ, in situ confining stress σ_{c}, the Biot coefficient β, the effective stress dependence coefficient α, and initial permeability k₀ may then be input into Equation (2) to determine in situ permeability k of in situ rock (200a, b).

In situ permeability k of in situ rock (200a, b) may be determined for in situ rock (200a, b) at various depths, horizontal positions, and times within the subterranean region of interest (100). In situ permeabilities k may then be used to predict a hydrocarbon production rate. In turn, predicted hydrocarbon production rates may be used, at least in part, to determine a production management plan.

A production management plan may define and organize the activities associated with producing hydrocarbons from a hydrocarbon reservoir (106). For example, a production management plan may define how rapidly to produce hydrocarbons for various time intervals for each well (514) over the lifetime of each well (514). A production management plan may also define when and where to drill new wells (514) and how to complete the new wells (514) that penetrate the hydrocarbon reservoir (106). Further, a production management plan may define when, where, and how to stimulate existing and new wells (514). Further still, a production management plan may define when to abandon a well (514).

FIG. 9 depicts a block diagram of a computer system (902) used to provide computational functionalities associated with described algorithms, methods, functions, processes, flows, and procedures as described in this disclosure, according to one or more embodiments. The illustrated computer (902) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (902) may include a computer that includes an input device, such as a keypad, keyboard, touch screen, or other device that can accept user information, and an output device that conveys information associated with the operation of the computer (902), including digital data, visual, or audio information (or a combination of information), or a GUI.

The computer (902) can serve in a role as a client, network component, a server, a database or other persistency, or any other component (or a combination of roles) of a computer system for performing the subject matter described in the instant disclosure. The illustrated computer (902) is communicably coupled with a network (930). In some implementations, one or more components of the computer (902) may be configured to operate within environments, including cloud-computing-based, local, global, or other environment (or a combination of environments).

At a high level, the computer (902) is an electronic computing device operable to receive, transmit, process, store, or manage data and information associated with the described subject matter. According to some implementations, the computer (902) may also include or be communicably coupled with an application server, e-mail server, web server, caching server, streaming data server, business intelligence (BI) server, or other server (or a combination of servers).

The computer (902) can receive requests over network (930) from a client application (for example, executing on another computer (902)) and responding to the received requests by processing the said requests in an appropriate software application. In addition, requests may also be sent to the computer (902) from internal users (for example, from a command console or by other appropriate access method), external or third-parties, other automated applications, as well as any other appropriate entities, individuals, systems, or computers.

Each of the components of the computer (902) can communicate using a system bus (903). In some implementations, any or all of the components of the computer (902), both hardware or software (or a combination of hardware and software), may interface with each other or the interface (904) (or a combination of both) over the system bus (903) using an application programming interface (API) (912) or a service layer (913) (or a combination of the API (912) and service layer (913). The API (912) may include specifications for routines, data structures, and object classes. The API (912) may be either computer-language independent or dependent and refer to a complete interface, a single function, or even a set of APIs. The service layer (913) provides software services to the computer (902) or other components (whether or not illustrated) that are communicably coupled to the computer (902). The functionality of the computer (902) may be accessible for all service consumers using this service layer. Software services, such as those provided by the service layer (913), provide reusable, defined business functionalities through a defined interface. For example, the interface may be software written in JAVA, C++, or other suitable language providing data in extensible markup language (XML) format or another suitable format. While illustrated as an integrated component of the computer (902), alternative implementations may illustrate the API (912) or the service layer (913) as stand-alone components in relation to other components of the computer (902) or other components (whether or not illustrated) that are communicably coupled to the computer (902). Moreover, any or all parts of the API (912) or the service layer (913) may be implemented as child or sub-modules of another software module, enterprise application, or hardware module without departing from the scope of this disclosure.

The computer (902) includes an interface (904). Although illustrated as a single interface (904) in FIG. 9, two or more interfaces (904) may be used according to particular needs, desires, or particular implementations of the computer (902). The interface (904) is used by the computer (902) for communicating with other systems in a distributed environment that are connected to the network (930). Generally, the interface (904) includes logic encoded in software or hardware (or a combination of software and hardware) and operable to communicate with the network (930). More specifically, the interface (904) may include software supporting one or more communication protocols, such as the Wellsite Information Transfer Specification (WITS) protocol, associated with communications such that the network (930) or interface's hardware is operable to communicate physical signals within and outside of the illustrated computer (902).

The computer (902) includes at least one computer processor (905). Although illustrated as a single computer processor (905) in FIG. 9, two or more processors may be used according to particular needs, desires, or particular implementations of the computer (902). Generally, the computer processor (905) executes instructions and manipulates data to perform the operations of the computer (902) and any algorithms, methods, functions, processes, flows, and procedures as described in the instant disclosure.

The computer (902) also includes a memory (906) that holds data for the computer (902) or other components (or a combination of both) that can be connected to the network (930). For example, memory (906) can be a database storing data consistent with this disclosure. Although illustrated as a single memory (906) in FIG. 9, two or more memories may be used according to particular needs, desires, or particular implementations of the computer (902) and the described functionality. While memory (906) is illustrated as an integral component of the computer (902), in alternative implementations, memory (906) can be external to the computer (902).

The application (907) is an algorithmic software engine providing functionality according to particular needs, desires, or particular implementations of the computer (902), particularly with respect to functionality described in this disclosure. For example, application (907) can serve as one or more components, modules, applications, etc. Further, although illustrated as a single application (907), the application (907) may be implemented as multiple applications (907) on the computer (902). In addition, although illustrated as integral to the computer (902), in alternative implementations, the application (907) can be external to the computer (902).

There may be any number of computers (902) associated with, or external to, a computer system containing a computer (902), wherein each computer (902) communicates over network (930). Further, the term “client,” “user,” and other appropriate terminology may be used interchangeably as appropriate without departing from the scope of this disclosure. Moreover, this disclosure contemplates that many users may use one computer (902), or that one user may use multiple computers (902).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method comprising:
obtaining, from a subterranean region of interest, a rock sample having a rock type;
defining a sequence of pore pressure, confining stress (PPCS) pairs such that a sequence of effective stresses monotonically changes;
determining, using a computer processor, a sequence of permeabilities by subjecting the rock sample to the sequence of PPCS pairs;
determining, using the computer processor, a relationship between the sequence of PPCS pairs and the sequence of permeabilities;
determining, using the computer processor, a parameter using the relationship and a permeability model, wherein the permeability model comprises the parameter; and
determining, using the computer processor, an in situ permeability for an in situ rock in the subterranean region of interest using, at least in part, the parameter and the permeability model, wherein the in situ rock is of the rock type.

2. The method of claim 1, wherein the rock sample comprises source rock.

3. The method of claim 1, wherein the rock type comprises shale.

4. The method of claim 1, further comprising:
determining a hydrocarbon production rate based, at least in part, on the in situ permeability; and
determining a production management plan based, at least in part, on the hydrocarbon production rate.

5. The method of claim 1, wherein obtaining the rock sample further comprises:
cutting the rock sample;
drying the rock sample; and
pre-stressing the rock sample.

6. The method of claim 1, wherein the rock sample is subjected to the sequence of PPCS pairs using a hydrostatic permeability system.

7. The method of claim 1, wherein pore pressures among the sequence of PPCS pairs are selected to minimize Knudsen diffusion.

8. The method of claim 7, wherein the pore pressures among the sequence of PPCS pairs are greater than 10 megapascals.

9. The method of claim 1, wherein determining the sequence of permeabilities comprises a pressure pulse decay method.

10. The method of claim 9, wherein the pressure pulse decay method comprises:
obtaining a permeability-pressure model;
subjecting a test sample to a PPCS pair;
generating a pressure pulse;
determining a first transient pressure due to the pressure pulse;
determining a second transient pressure due to the pressure pulse; and
determining a permeability by fitting, in part, the first transient pressure and the second transient pressure to the permeability-pressure model.

11. The method of claim 1, wherein the parameter comprises a Biot coefficient.

12. The method of claim 1, wherein determining the relationship and determining the parameter comprises:
for each unique pore pressure among the sequence of PPCS pairs:
fitting a linear line to confining stresses versus a natural logarithm of the sequence of permeabilities associated to each unique pore pressure, and
determining a slope from the linear line; and
determining the parameter as an average slope from a plurality of the determined slopes.

13. The method of claim 1, wherein determining the relationship and the parameter comprises:
for each unique PPCS difference among the sequence of PPCS pairs:
fitting a linear line to pore pressures versus a natural logarithm of the sequence of permeabilities associated to each unique PPCS difference, and
determining an intercept from the linear line;
fitting a new linear line to each unique PPCS difference and the intercept for a plurality of the unique PPCS differences; and
determining the parameter as a slope of the new linear line.

14. The method of claim 1, wherein determining the relationship and the parameter comprises:
assuming a first parameter;
fitting an exponential line to the sequence of effective stresses and the sequence of permeabilities; and
determining a second parameter from the exponential line.

15. A system comprising:
a hydrostatic permeability system configured to subject a rock sample to a sequence of pore pressure, confining stress (PPCS) pairs; and
a computer system configured to:
receive the sequence of PPCS pairs such that a sequence of effective stresses monotonically changes,
determine a sequence of permeabilities following the rock sample being subjected to the sequence of PPCS pairs using the hydrostatic permeability system,
determine a relationship between the sequence of PPCS pairs and the sequence of permeabilities,
determine a parameter using the relationship and a permeability model, wherein the permeability model comprises the parameter, and
determine an in situ permeability for an in situ rock in a subterranean region of interest using, at least in part, the parameter and the permeability model, wherein the in situ rock is of a rock type.

16. The system of claim 15, further comprising a rock sample extraction tool configured to obtain the rock sample from the subterranean region of interest, wherein the rock sample is of the rock type.

17. The system of claim 16, wherein the rock sample extraction tool comprises a coring system.

18. The system of claim 15, wherein the hydrostatic permeability system comprises:
a pressure generator configured to apply a confining stress to the rock sample; and
a gas pump system configured to apply a pore pressure to the rock sample, wherein the gas pump system comprises:
an upstream reservoir, and
a downstream reservoir.

19. The system of claim 18, wherein the gas pump system is configured to emit a pressure pulse.

20. The system of claim 18, wherein the gas pump system houses helium.
